(19) United States
(12) Patent Application Publication
(10) Pub. No.: US 2004/0064028 A1
(43) Pub. Date: Apr. 1, 2004

(54) Head-fixing device for an imaging medical examination device, and examination device using same

(75) Inventor: Michael Deimling, Moehrendorf (DE)

Correspondence Address: SCHIFF HARDIN, LLP, PATENT DEPARTMENT, 6600 SEARS TOWER, CHICAGO, IL, 60606-6473, US

(73) Assignee: Siemens Aktiengesellschaft

(21) Appl. No.: 10/670,041

(22) Filed: Sep. 24, 2003

(30) Foreign Application Priority Data
Sep. 30, 2002 (DE) 10245487.6

(51) Int. Cl. A61B 5/05

(52) U.S. Cl. 600/407

(57) ABSTRACT

In a head-fixing device for a medical imaging examination device, an acoustic signal emitter is integrated into the head-fixing device. A fixing cushion is shaped such that when fixing the head it forms a chamber enclosing the ear. In this chamber, sound is transmitted pneumatically via an air canal.

### BACKGROUND OF THE INVENTION

1. Field of the Invention

The invention concerns a head-fixing device for a medical imaging examination device.

2. Description of the Prior Art

Controlled positioning and fixing of a patient in an imaging medical examination device are important for many examinations that require the position of the body part to be examined to be reproducible and unchanging during the examination. Typically, for this reason fixing cushions are pressed, for example on the head of the patient, by means of a pressing device. Many examinations extend over a long period of time, such that in the positioning appropriate attention must be given to the comfort of the patient and communication with the patient, as needed, should be possible. The acoustic environment must be considered, since the examinations ensue for the most part in closed examination devices that hamper communication, or considerable background noises are present that are generated from noise sources during the examination, for example the generation of magnetic fields in magnetic resonance tomography devices.

One-time ear plugs are effective noise-protection means to protect the patient, but their use is inconvenient, and they cause expense and waste, and require valuable time in the preparation of the patient. An alternative that additionally enables communication with the patient is a headphone that is put on the patient. A headphone, however, limits the possible positions of the fixing cushions and thus leads to a worsening of the fixing of the head. An effective use of the headphone is only the case wherein it can be applied in a soundproof manner to the head, and wherein it does not shift during the examination. In longer examinations, these conditions unfortunately are not always guaranteed.

A magnetic resonance examination device is known from PCT Application WO 01/22108 that has (attached to the patient positioning device) an arced carrier element on which for example, a speaker, a microphone, and a mirror are attached.

An audio system for patients in magnetic resonance examination devices is known from U.S. Pat. No. 5,277,184 that has a pneumatic, non-magnetic transducer that converts electrical signals into noise by means of a piezoelectric speaker.

### SUMMARY OF THE INVENTION

An object of the present invention is to improve communication with a patient positioned by means of a head-fixing device.

This object is inventively achieved by a head-fixing device for an imaging medical examination apparatus wherein an acoustic signal emitter is integrated into the head-fixing device. This has the advantage that a fixing cushion can be pressed, unhindered, over a large area on the head by the acoustic signal emitter. The result is a very good fixing of the head with a simultaneous good positioning of the acoustic signal emitter. The signal emitter also is fixed in position at the head with the fixing of the position of the head itself, and cannot shift. Consistent and controllable acoustic conditions for the patient thus are achieved. In the head-fixing device according to the invention the head fixing structure and the acoustic signal emitter are combined in a structural unit.

In an embodiment, the fixing of the head is achieved with an adjustable arrangement. This simplifies the application of the head-fixing device and makes it possible, with the use of light pressure of the adjustable arrangement on the head, to create a chamber around the ear of the patient in which the acoustic signals are emitted.

In a further embodiment fixing cushions are pressed on the head with a threaded spindle. A threaded spindle is simple to adjust and easily adaptable to the conditions in the imaging medical examination device.

In another embodiment, the head-fixing device has additional sound protection means. For example, in the fixing of the head, a cushion adapted to the shape of the head can circularly enclose the ear of the patient for noise isolation. In a version of this embodiment, the fixing cushion can be pressed in a soundproof manner on the head with a pressing device, and thus form a chamber surrounding the ear. In this manner, the patient is acoustically isolated from the noise sources of the medical imaging examination device.

In another embodiment, the acoustic signal emitter is fashioned as a type of headphone. For example, two noise-isolating cushions are symmetrically put over both ears in the fixing of a head. Sound to be transmitted, for example music or instructions of the personnel, are conducted to a chamber surrounding the ear and fed into this chamber by the acoustic signal emitter. This has the advantage of a symmetrical assembly that offers good acoustics and, for example, enables stereo listening.

The acoustic signal emitter in the head-fixing device can be implemented in the manner of a stethoscope headphone. This means that only a pneumatic connection is necessary in the chamber enclosing the ear.

In another embodiment, the acoustic signal emitter can be connected with a sound source by an air tube. In a simple manner, acoustic signals are transmitted via the air tube to the patient in the examination device. This has the advantage that the sound transmission is not dependent on electrical or mechanical components, but rather ensues via the air in the air tube alone. This has the particular advantage that the head-fixing device is designed so that it does not produce distortions in a magnetic resonance tomography device.

A further improvement in the signal transmission quality is achieved in an embodiment wherein the pneumatic supply line of the acoustic signal is isolated from electromagnetic interferences by a shielding from external acoustic interference sources or from an electromagnetic supply line. A further advantage of such a shielding is that no interferences are generated in the signal acquisition by the magnetic resonance tomography device as a result of the head-fixing device according to the invention. This is possible given electrical signal pathways and must likewise by prevented by a shielding.

In another embodiment, the air tube is integrated into the patient positioning device, such that no interfering components are open in the examination device. This has the additional advantage that the air tube is acoustically isolated, meaning that no noise can intrude from surrounding noise sources into the air tube.

Furthermore, the above object is achieved by a medical examination device with a head-fixing device of the type described above.

### DESCRIPTION OF THE DRAWINGS

FIG. 1 shows an inventive head-fixing device that is pressed on the head of a patient to be examined by means of a threaded spindle and that has openings to pneumatically transmit acoustic signals.

FIG. 2 a section through a head-fixing shell of the device of FIG. 1 having recesses in the area of the ears of a positioned patient.

### DESCRIPTION OF THE PREFERRED EMBODIMENTS

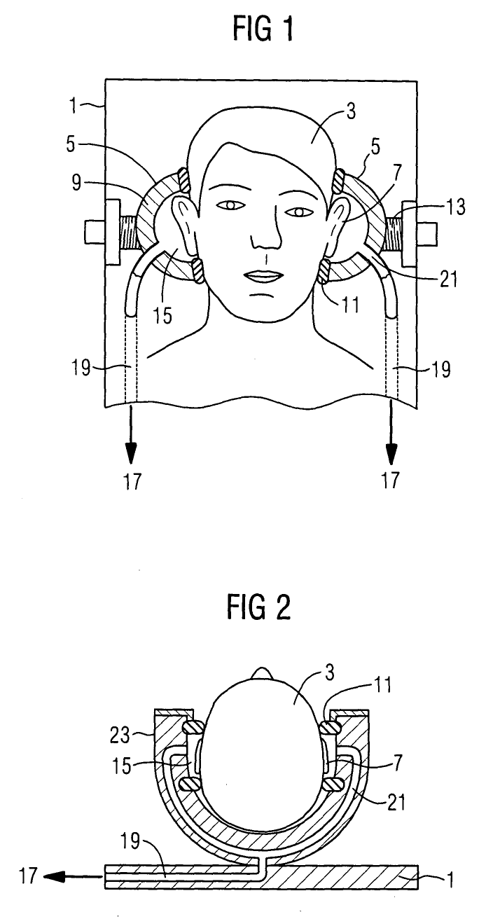

In FIG. 1, the assembly of a head-fixing device is schematically shown. A patient lies on the patient positioning device 1. His or her head 3 is positioned by the head-fixing device and thereby is fixed in position. The head-fixing device has two ear shells that respectively surround the ears 7 of the patient. The ear shells 5 have a firm fixing cushion 9 that is applied annularly on the head around each ear 7. A soundproof protection cushion 11 is located in the contact area. Both fixing cushions 9 are pressed on the head by threaded spindles 13. The threaded spindles 13 are attached to the patient positioning device 1. The fixing cushions 9 create two chambers 15 that surround the ears 7. Acoustic signals, for example music or information from the personnel attending the examination, are emitted from a sound source 17 and fed into one or, in the case of a stereo transmission, two air tubes 19. The air tubes 19 are embedded in the patient positioning device 1. The material surrounding the air tubes 19, for example the plastic of a patient bed, serves as sound protection means and prevents significant coupling of surrounding noise into the air tubes 19. A complicated design or protection of the air tubes 19 thus is not needed. The air tubes 19 emerge from the patient positioning device 1 In the area of the head and are connected with air channels 21 of the fixing cushions 9 that conduct the sound into the chamber 15 surrounding the ear. This type of supply line resembles a stethoscope headphone.

In FIG. 2, a section through a head-fixing device according to the invention is shown that is based on a head-fixing shell 23. The head-fixing shell 23 is firmly connected with the patient positioning device 1. It has recesses in the area of the ears 7. The recesses are lined by an annular sound protection cushion 11, the sound protection cushion 11 forming a soundproof coupling to the head 3 of the patient. The recesses again form the chambers 15 into which acoustic signals, for example instructions of the attending personnel, are fed by air channels 21 embedded in the head-fixing shell. The air channels 21 are again connected with an air tube embedded in the patient positioning device 1 that for its part is connected with the sound source 17. The head-fixing shell can, by means of a clamp device (not shown), can effect a stronger fixing of the head, while simultaneously achieving improved soundproof termination of the chamber 15.

Although modifications and changes may be suggested by those skilled in the art, it is the intention of the inventor to embody within the patent warranted hereon all changes and modifications as reasonably and properly come within the scope of his contribution to the art.

I claim as my invention:

1. A device for fixing a head of a patient in a medical imaging examination device, comprising:
a fixing arrangement adapted to interact with a head of a patient to fix a position of the head; and
an acoustic signal emitter integrated into said fixing arrangement adapted to emit an acoustical signal perceivable by the patient.

2. A device as claimed in claim 1 wherein said fixing arrangement is adjustable relative to the head of the patient.

3. A device as claimed in claim 2 wherein said fixing arrangement comprises a frame and fixing elements adapted to interact with the head of the patient, and respective threaded spindles, mounted to the frame, for adjusting the respective fixing elements.

4. A device as claimed in claim 1 wherein said fixing arrangement comprises soundproofing for preventing sounds originating from an exterior of the fixing device to be perceivable by the patient.

5. A head fixing device as claimed in claim 4 wherein said fixing arrangement comprises ear enclosures, and wherein said ear enclosures comprise soundproofing material.

6. A device as claimed in claim 1 wherein said acoustic signal emitter is a headphone.

7. A device as claimed in claim 1 wherein said acoustic signal emitter is a stethoscope-type headphone with pneumatic sound transmission.

8. A device as claimed in claim 1 further comprising a sound source connected to said acoustic signal emitter.

9. A device as claimed in claim 1 comprising an air tube connecting said sound source to said acoustic signal emitter.

10. A device as claimed in claim 1 wherein said medical imaging examination device has a patient positioning device, and wherein said air tube is adapted to be integrated into said patient positioning device.

11. A device as claimed in claim 1 wherein said fixing arrangement and said acoustic signal emitter are compatible for use in magnetic resonance tomography.

12. A device as claimed in claim 1 further comprising shielding for said fixing arrangement that suppresses electric magnetic transmission into and out of said fixing arrangement.

13. A device as claimed in claim 1 wherein said fixing arrangement and said acoustic sound emitter are substantially free of electrically conductive materials.

14. A device as claimed in claim 1 wherein said fixing arrangement and said acoustic signal emitter are substantially free of magnetic materials.

15. A medical imaging examination device comprising:
a patient positioning device adapted to receive a patient thereon;
a fixing arrangement disposed on said patient positioning device adapted to interact with a head of the patient to fix a position of the head of the patient on the patient positioning device; and
an acoustic signal emitter integrated into said fixing arrangement.

16. A medical imaging examination device as claimed in claim 15 comprising a sound-transmitting channel proceeding from said acoustic signal emitter and integrated in said patient positioning device.
